(19) United States
(12) Patent Application Publication
(10) Pub. No.: US 2006/0057629 A1
(43) Pub. Date: Mar. 16, 2006

(54) Device for injecting PCR solution into PCR channels of PCR chip, and PCR chip unit including the device

(76) Inventor: Min-soo Kim, Seoul (KR)

Correspondence Address: CANTOR COLBURN, LLP, 55 GRIFFIN ROAD SOUTH, BLOOMFIELD, CT, 06002, US

(21) Appl. No.: 11/226,545

(22) Filed: Sep. 14, 2005

(30) Foreign Application Priority Data
Sep. 16, 2004 (KR) 10-2004-0074192

(51) Int. Cl. C12Q 1/68; C12P 19/34; C12M 1/34

(52) U.S. Cl. 435/6; 435/91.2; 435/287.2

(57) ABSTRACT

Provided is a device for injecting a PCR solution into PCR channels of a PCR chip. The device includes: first through-holes corresponding to each of the inlets of the PCR chip in which PCR channels are formed; and second through-holes corresponding to each of the outlets of the PCR chip. The first through-holes are aligned with each of the corresponding inlets of the PCR chip and the second through-holes are aligned with each of the corresponding outlets of the PCR chip when the PCR solution is injected so that the PCR solution can flow into the inside of the PCR channels through the first through-holes or the second through-holes. The device slides to seal the inlets and the outlets of the PCR chip after the PCR solution is injected.

This application claims the priority of Korean Patent Application No. 10-2004-0074192, filed on Sep. 16, 2004 in the Korean Intellectual Property Office, the disclosure of which is incorporated herein in its entirety by reference.

### BACKGROUND OF THE INVENTION

1. Field of the Invention

The present invention relates to a device for injecting a PCR solution into PCR channels, and a PCR chip unit including the device.

2. Description of the Related Art

A PCR solution is manually injected into an inlet and/or outlet of a conventional PCR chip using equipment such as a pipette. During the manual operation, confusion may arise between PCR channels when using a multi-channel PCR chip. In addition, once the PCR solution is injected, a PCR solution can be lost while performing PCR due to, for example, evaporation. Therefore, to prevent the lost of the PCR solution, a surface of the PCR chip on which the inlet and/or outlet is formed must be taped or sealed with a sealing material. Consequently, according to the conventional art, the process of manually injecting the PCR solution and the process of sealing the inlet and/or outlet with, for example, the tape after injecting the PCR solution must be performed.

Although the conventional art can be used in a single channel PCR chip, it is inconvenient to use in a multi-channel PCR chip. Therefore, a method and apparatus for easily sealing an inlet and/or outlet of a PCR chip in the multi-channel PCR chip after accurately injecting a PCR solution are required.

The inventors of the present application have completed the present invention while researching the method of simply and accurately injecting the PCR solution into the multi-channel PCR chip.

### SUMMARY OF THE INVENTION

The present invention provides a device for simply and accurately injecting a PCR solution into each PCR channels of a PCR chip.

The present invention provides a PCR chip unit including the device.

According to an aspect of the present invention, there is provided a device for injecting a PCR solution into PCR channels of a PCR chip, the device including: first through-holes corresponding to each of the inlets of the PCR chip in which PCR channels are formed; and second through-holes corresponding to each of the outlets of the PCR chip. The first through-holes are aligned with each of the corresponding inlets of the PCR chip and the second through-holes are aligned with each of the corresponding outlets of the PCR chip when the PCR solution is injected so that the PCR solution can flow into the inside of the PCR channels through the first through-holes or the second through-holes, and the device slides to seal the inlets and the outlets of the PCR chip after the PCR solution is injected.

In an embodiment of the present invention, on the device of the present invention, first protrusions corresponding to each of the inlets of the PCR chips and second protrusions corresponding to each of the outlets of the PCR chips are formed, the first and second protrusions sliding after the PCR solution is injected. The first and second protrusions are made of elastic elements and seal the corresponding inlets and outlets.

In another embodiment of the present invention, the device of present invention may further include fixing elements which couple an upper housing to a bottom housing to fix the device at a predetermined location of the PCR chip. The fixing elements may be ratchets.

In another embodiment of the present invention, grooves are formed between the first through-holes or between the second through-holes of the present device. By using these grooves, when the PCR mixtures are injected into the PCR channels through the through-holes, the PCR mixtures are prevented from flowing into different through-holes adjacent to the target through-holes.

According to another aspect of the present invention, there is provided a PCR chip unit including, a PCR chip in which PCR channels are formed; a bottom housing disposed below the PCR chip to fix the bottom portion of the PCR chip; a device for injecting a PCR solution into the PCR channels of the PCR chip; and an upper housing disposed on the top of the device to fix the device. The device includes: first through-holes corresponding to each of the inlets of the PCR chip in which PCR channels are formed; and second through-holes corresponding to each of the outlets of the PCR chip. The first through-holes are aligned with each of the corresponding inlets of the PCR chip and the second through-holes are aligned with each of the corresponding outlets of the PCR chip when the PCR solution is injected so that the PCR solution can flow into the inside of the PCR channels through the first through-holes or the second through-holes. The device slides to seal the inlets and the outlets of the PCR chip after the PCR solution is injected.

In an embodiment of the present invention, on the device, first protrusions corresponding to each of the inlets of the PCR chips and second protrusions corresponding to each of the outlets of the PCR chips are formed, the first and second protrusions sliding after the PCR solution is injected. The first and second protrusions are made of elastic elements and seal the corresponding inlets and outlets.

In an embodiment of the present invention, the device further includes: fixing elements which couple the upper housing to a bottom housing to fix the device at a predetermined location of the PCR chip. The fixing elements may be ratchets.

In another embodiment of the present invention, in the device, grooves are formed between the first through-holes or between the second through-holes. By using these grooves, when the PCR mixtures are injected into the PCR channels through the through-holes, the PCR mixtures are prevented from flowing into different through-holes adjacent to the target through-holes.

### BRIEF DESCRIPTION OF THE DRAWINGS

The above and other features and advantages of the present invention will become more apparent by describing in detail exemplary embodiments thereof with reference to the attached drawings in which:

FIG. 1 is a plan view of a device for injecting a PCR solution into PCR channels of a PCR chip according to an embodiment of the present invention;

FIG. 2 is a diagram illustrating a multi-channel PCR chip with six PCR channels formed thereon, coupled to a bottom housing according to an embodiment of the present invention;

FIG. 3 is a diagram illustrating the device fixed on the top of the multi-channel PCR chip fixed on the bottom housing in FIG. 2;

FIG. 4 is a diagram illustrating the device fixed to the multi-channel PCR chip and an upper housing by placing the upper housing on top of the device as illustrated in FIG. 3 and coupling the upper housing to the bottom housing.

FIG. 5 is a diagram illustrating the device that has relatively slid in the horizontal direction with respect to the upper housing, sealing inlets and/or outlets; and

FIGS. 6 and 7 are cross-sections of the device taken along the line 2-2′ in FIGS. 4 and 5.

### DETAILED DESCRIPTION OF THE INVENTION

The present invention will now be described more fully with reference to the accompanying drawings, in which exemplary embodiments of the invention are shown, and thus the drawings should not be used to limit the scope of the present invention.

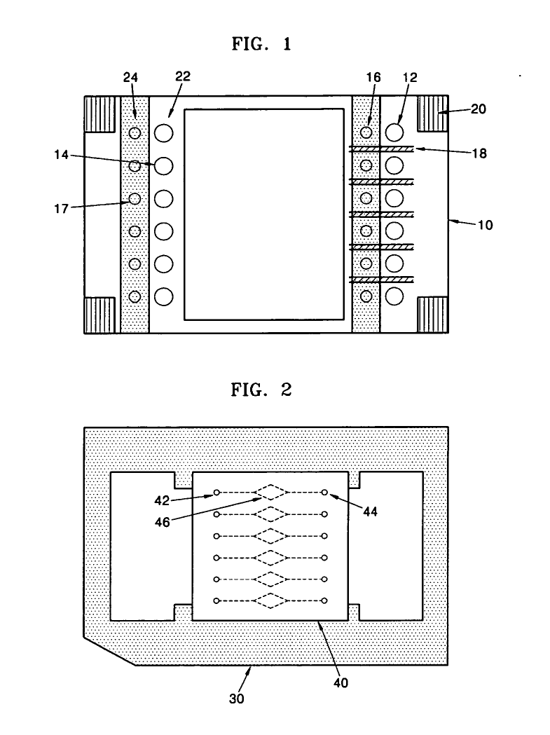

FIG. 1 is a plan view of a device 10 for injecting a PCR solution into PCR channels of a PCR chip according to an embodiment of the present invention. The device 10 includes first through-holes 12 each corresponding to inlets of the PCR chip in which PCR channels are formed, and second through-holes 14 each corresponding to outlets of the PCR chip. When injecting the PCR solution, the first though-holes 12 are aligned with corresponding inlets of the PCR chip and the second through-hole 14 are aligned with corresponding outlets of the PCR chip so that the PCR solution can be injected into the PCR channels through the first through-holes 12 or the second through-holes 14. In addition, when the device 10 slides by receiving a horizontal force after the PCR solution is injected, the inlets and outlets of the PCR chip are sealed. In the present embodiment, the first through-holes 12 and the second through-holes 14 may or may not have the same dimension. In addition, the PCR solution can be injected through the first through-holes 12 or the second through-holes 14.

The device 10 may further selectively include first protrusions 16 and second protrusions 17 respectively adjacent to the first through-holes 12 and the second through-holes 14 on the bottom surface of the device 10. The first and second protrusions 16 and 17 may be made of an elastic material on a region 24 also made of an elastic material. Besides the region 24 made of the elastic material, a region 22 is made of a non-elastic material. When a force is applied to the device 10 in the horizontal direction after the PCR solution is injected, the device 10 slides and seals the inlets and outlets of the PCR chip.

The device 10 may further selectively include fixing elements 20 disposed on the top of the device 10. The fixing elements 20 are coupled to an upper housing and fixed to the upper housing. The fixing elements 20 may be a ratchet.

The device 10 may further selectively include grooves 18 formed between the first through-holes 12 or between the second through-holes 14 on the bottom of the device 10. When the PCR solution is injected through the first through-holes 12 or the second through-holes 14, the PCR solution flowing into adjacent through-holes can be prevented by the grooves 18.

FIGS. 2 through 7 are views illustrating a process of assembling the device 10 into a PCR chip unit, and the effects thereof. FIG. 2 is a diagram illustrating a multi-channel PCR chip 40 with six PCR channels formed therein, coupled to a bottom housing 30. The multi-channel PCR chip 40 is fixed to the bottom housing 30 by a fixing element. The fixing element may be a fixing element including a meshing element. Channels each having inlets 44 and outlets 42 are formed in the multi-channel PCR chip 40. PCR is usually performed in chambers 46 inside the channels.

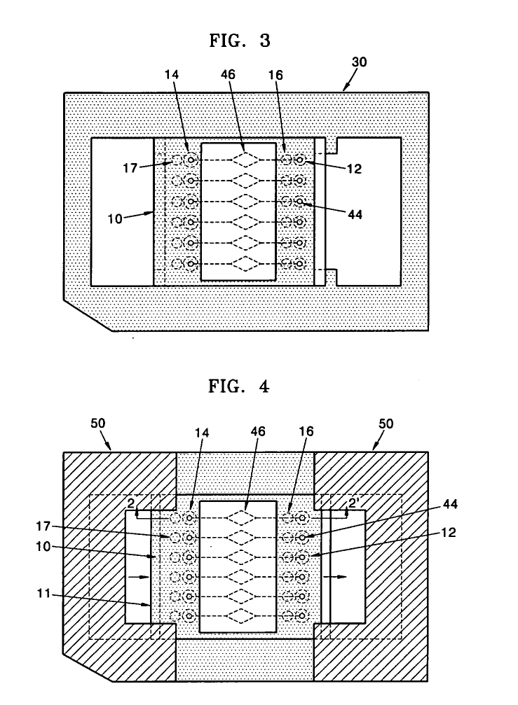

FIG. 3 is a diagram illustrating the device 10 fixed on the top of the multi-channel PCR chip 40 fixed on the bottom housing 30 in FIG. 2. The first through-holes 12 of the device 10 are disposed to correspond to the inlets 44 of the multi-channel PCR chip 40, and the second through-holes 14 of the device 10 are disposed to correspond to the outlets 42 of the multi-channel PCR chip 40. Therefore, the first through-holes 12 are aligned with the corresponding inlets 44 and the second through-holes 14 are aligned with the corresponding outlets 42, and thus the injected PCR solution can flow into the chambers 46 of the multi-channel PCR chip 40. The device 10 may selectively include first protrusions 16 disposed adjacent to the first through-holes 12 and second protrusions 17 disposed adjacent to the second through-holes 14 on the bottom surface of the device 10 facing the top surface of the multi-channel PCR chip 40.

FIG. 4 is a diagram illustrating the device 10 fixed to the multi-channel PCR chip 40 and the upper housing 50 by placing the upper housing 50 on the top of the device 10 illustrated in FIG. 3 and then coupling the upper housing 50 to the bottom housing 30. By coupling the upper housing 50, the device 10 is coupled to be able to slide between the multi-channel PCR chip 40 and the upper housing 50. The PCR solution flows into the chambers 46 via the first through-holes 12 and the inlets 44 aligned with the first through-holes 12. Alternatively, the PCR solution flows into the chambers 46 via the second through-holes 14 and the outlets 42 aligned with the second through-holes 14. The device 10 includes an exposed portion 11 to which a force is manually or automatically applied in the horizontal direction as indicated in an arrow in FIG. 4. The device 10 can relatively slide with respect to the multi-channel PCR chip 40 on the bottom of the device 10 and the upper housing 50 on the top of the device 10. The PCR solution flows into the chambers 46 by sequentially flowing through the first or the second through-holes 12 and 14 and the outlets 44 or the outlets 42. Next, by applying a force in the horizontal direction to the exposed portion 11, the device 10 relatively slides with respect to the upper housing 50 and the multi-channel PCR chip 40, thereby sealing the inlets 44 and/or the outlets 42.

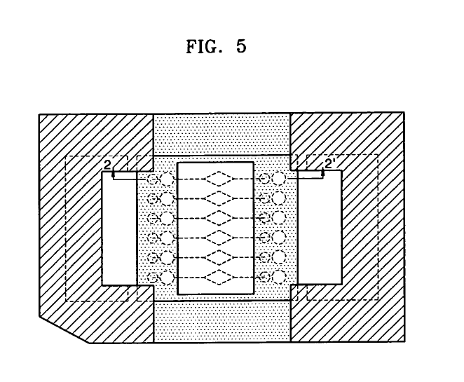

FIG. 5 is a diagram illustrating the device 10 that has relatively slid in the horizontal direction with respect to the top housing 50 and the multi-channel PCR chip 40. As shown, PCR can be performed by repeatedly applying heat to the PCR channels when the PCR solution fills the chambers 46 and sealed by the device 10.

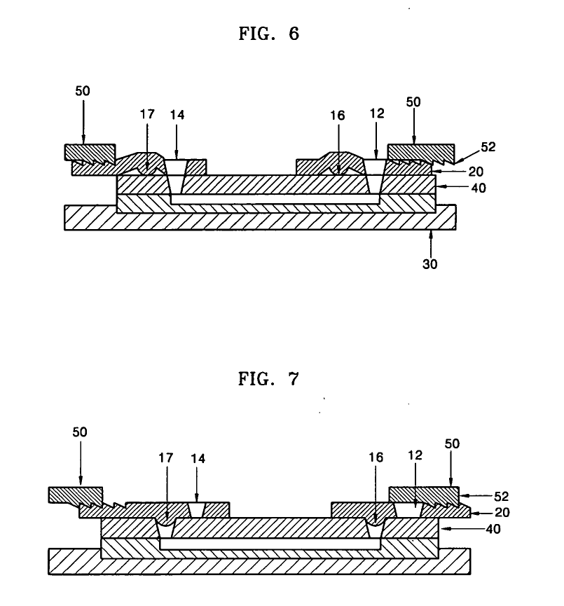

FIGS. 6 and 7 are cross-sections of the device 10 taken along the lines 2-2′ illustrated in FIGS. 4 and 5, respectively. As shown in FIGS. 6 and 7, by injecting the PCR solution through the first through-holes 12 and/or second through-holes 14, the PCR solution flows into the chambers 46 inside the multi-channel PCR chip 40. Next, by applying a force to the exposed portion 11, the device 10 slides to seal the inlets 44 and the outlets 42 and be fixed.

The device 10 may selectively include protrusions 16 and 17 facing the top surface of the multi-channel PCR chip 40, disposed adjacent to the first through-holes 12 and the second through-holes 14, respectively. The protrusions 16 and 17 slide to effectively seal the inlets 44 and/or outlets 42.

The device 10 may selectively include fixing elements to couple the upper housing 50 to the device 10. The fixing elements may be ratchets 50 and 52.

The size, structure, and material of the device 10 are not limited to the ones illustrated in the drawings, and can be configured in various sizes, structures, and materials.

According to a device for injecting a PCR solution into PCR channels and a PCR chip unit including the device, the PCR solution can be injected into the correct PCR channel without confusing with different PCR channels, and the fixing and sealing processes of the PCR chip unit can be performed through a simple method. Therefore, processes of adhering tape or sealing inlets and outlets of a PCR chip after injecting a PCR solution in the conventional art do not need to be performed.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A device for injecting a PCR solution into PCR channels of a PCR chip, the device comprising:
first through-holes corresponding to each of the inlets of the PCR chip in which PCR channels are formed; and
second through-holes corresponding to each of the outlets of the PCR chip,
wherein the first through-holes are aligned with each of the corresponding inlets of the PCR chip and the second through-holes are aligned with each of the corresponding outlets of the PCR chip when the PCR solution is injected so that the PCR solution can flow into the inside of the PCR channels through the first through-holes or the second through-holes, and
the device slides to seal the inlets and the outlets of the PCR chip after the PCR solution is injected.

2. The device of claim 1, wherein on the device, first protrusions corresponding to each of the inlets of the PCR chips and second protrusions corresponding to each of the outlets of the PCR chips are formed, the first and second protrusions sliding after the PCR solution is injected,
wherein the first and second protrusions are made of elastic elements and seal the corresponding inlets and outlets.

3. The device of claim 1, further comprising: fixing elements which couple the device to an upper housing to fix the device at a predetermined location of the PCR chip.

4. The device of claim 3, wherein the fixing elements are ratchets.

5. The device of claim 1, wherein grooves are formed between the first through-holes or between the second through-holes.

6. A PCR chip unit comprising:
a PCR chip in which PCR channels are formed;
a bottom housing disposed below the PCR chip to fix the bottom portion of the PCR chip;
a device for injecting a PCR solution into the PCR channels of the PCR chip, the device including:
first through-holes corresponding to each of the inlets of the PCR chip in which PCR channels are formed; and
second through-holes corresponding to each of the outlets of the PCR chip,
wherein the first through-holes are aligned with each of the corresponding inlets of the PCR chip and the second through-holes are aligned with each of the corresponding outlets of the PCR chip when the PCR solution is injected so that the PCR solution can flow into the inside of the PCR channels through the first through-holes or the second through-holes, and
the device slides to seal the inlets and the outlets of the PCR chip after the PCR solution is injected; and
an upper housing disposed on the top of the device to fix the device.

7. The PCR chip unit of claim 6, wherein on the device, first protrusions corresponding to each of the inlets of the PCR chips and second protrusions corresponding to each of the outlets of the PCR chips are formed, the first and second protrusions sliding after the PCR solution is injected,
wherein the first and second protrusions are made of an elastic element and seals the corresponding inlets and outlets.

8. The PCR chip unit of claim 6, wherein the device further comprises: fixing elements which couple the device to an upper housing to fix the device at a predetermined location of the PCR chip.

9. The PCR chip unit of claim 8, wherein the fixing elements are ratchets.

10. The PCR chip unit of claim 6, wherein in the device, grooves are formed between the first through-holes or between the second through-holes.
